(19) United States
(12) Patent Application Publication
(10) Pub. No.: US 2006/0088493 A1
(43) Pub. Date: Apr. 27, 2006

(54) Composition and process for the cosmetic treatment of keratin materials based on electrophilic monomers and non-silicone polymers

(76) Inventors: Gabin Vic, Venette (FR); Aude Livoreil, Paris (FR); Gaelle Brun, Paris (FR); Luc Gourlaouen, Asnieres (FR); Franck Giroud, Clichy (FR); Isabelle Rollat-Corvol, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,293

(22) Filed: Oct. 13, 2005

Related U.S. Application Data
(60) Provisional application No. 60/646,492, filed on Jan. 25, 2005.

(30) Foreign Application Priority Data
Oct. 13, 2004 (FR) 04 10807

(51) Int. Cl. A61K 8/73; A61K 8/81

(52) U.S. Cl. 424/70.13; 424/70.16

(57) ABSTRACT

The present patent application relates to a process for the cosmetic treatment of keratin materials, including keratin fibers such as the hair. The process comprises applying the composition to the keratin materials, wherein the composition comprises, in a cosmetically acceptable medium, at least one electrophilic monomer and at least one non-silicone polymer, the non-silicone polymer being chosen such that the composition gives, after drying, a film with a maximum peel force of greater than 1 newton. The application also relates to the composition.

This application claims benefit of U.S. Provisional Application No. 60/646,492, filed Jan. 25, 2005, the contents of which are incorporated herein by reference. This application also claims benefit of priority under 35 U.S.C. § 119 to French Patent Application No. 04 10807, filed Oct. 13, 2004, the contents of which are also incorporated by reference.

The present disclosure relates to the use for the cosmetic treatment of keratin materials, including keratin fibers such as the hair, of a non-tacky composition based on in-situ polymerizable monomers, wherein the composition comprises a cosmetically acceptable medium and at least one non-silicone polymer.

Another aspect of the present disclosure relates to a method of treating keratin materials, including keratin fibers such as the hair, comprising applying a composition to the keratin materials, wherein the composition comprises at least one electrophilic monomer and at least one non-silicone polymer.

A further aspect of the present disclosure relates to a composition comprising at least one electrophilic monomer and at least one non-silicone polymer.

According to the present disclosure, the term “keratin materials” means keratin fibers, which includes materials such as hair, eyelashes, and nails.

Cosmetic compositions based on silicones or polymers with high affinity for keratin materials, such as the hair, are generally used in order to modify their surface properties, such as, for example, to condition them and to give them sheen.

It is generally necessary to renew these treatments insofar as the conditioning agents have a tendency to become removed, especially on shampooing.

It is theoretically possible to increase the remanence of the polymer deposit by directly performing a free-radical polymerization of certain monomers on the hair.

However, the treatments thus obtained are cosmetically unacceptable. High degradation of the fiber is generally observed, probably associated with the polymerization initiators, and the treated hair is difficult to disentangle, to the point that it is nowadays sought to obtain cosmetic compositions that can give the hair softness and long-lasting sheen.

In addition, cosmetic products intended for treating the hair, such as those for obtaining shape and styling effects, generally use compositions that form a tacky film (which has surface adhesion) on the hair. In general, the feel of hair thus treated is particularly coarse and unpleasant. When the user passes a hand through the hair, some of the polymers can become deposited on the fingers. This transfer phenomenon leaves an impression of dirty hair. In addition, this tacky coating becomes eliminated immediately on washing the hair. It is thus necessary to reapply the product after each shampoo wash.

There is thus a desire for compositions which do not adhere to the fingers after application to the hair, which do not turn powdery and which give the hair good wash-fast cosmetic properties.

The inventors have just discovered, surprisingly, a composition having good wash-fast properties and which decreases adherence to the fingers after application to the hair, wherein the composition comprising, in a cosmetically acceptable medium, a mixture of at least one electrophilic monomer, as described in patent application FR 2 840 208, and at least one non-silicone polymer.

The inventors have observed that by applying a composition based on at least one electrophilic monomer and at least one non-silicone polymer to keratin fibers, a lubricating, shiny, soft remanent coating is formed in situ.

Moreover, the inventors have observed, surprisingly, that the hairs remain individualized and can be easily styled, and that the conditioning and sheen of the fibers are shampoo-fast.

A first aspect of the present disclosure relates to the use for the cosmetic treatment of keratin materials, including keratin fibers such as the hair, of a composition comprising, in a cosmetically acceptable medium, at least one electrophilic monomer and at least one non-silicone polymer, the at least one non-silicone polymer being chosen such that the composition gives, after drying, a film with a maximum peel force of greater than 1 newton.

Another aspect of the present disclosure relates to a process using a composition for the cosmetic treatment of keratin materials, wherein the composition comprises at least one electrophilic monomer and at least one non-silicone polymer.

A further aspect of the disclosure relates to a kit comprising a first composition containing at least one electrophilic monomer, which may be present, for example, in an amount ranging from 0.5% to 50% of the weight of the first composition, and optionally at least one anionic and/or free-radical polymerization inhibitor, which may be present, for example, in an amount ranging from 10 ppm to 5% of the weight of the first composition, and a second composition comprising, in a cosmetically acceptable medium, at least one non-silicone polymer, which may be present, for example, in an amount ranging from 0.001% to 5% of the weight of the second composition.

An additional aspect of the present disclosure relates to a cosmetic composition comprising at least one electrophilic monomer and at least one non-silicone fixing polymer.

Other aspects of the present disclosure will become apparent on reading the description and the examples that follow.

According to the present disclosure, the term “maximum peel force” means the maximum tensile force, measured using an extensometer, required to peel apart the 38 mm² respective surfaces of two rigid, inert, non-absorbent supports (A) and (B), placed facing each other; wherein the surfaces are precoated with the composition at a rate of 519 μg/mm², dried for 24 hours at 22° C. under a relative humidity of 50%, and then subjected for 20 seconds to a compression of 3 newtons and finally subjected for 30 seconds to traction at a speed of 20 mm/minute.

An extensometer may be used for this measurement, for example a machine such as the Lloyd model LR5K.

The rigid, inert, non-absorbent supports may be chosen from those consisting of polyethylene, polypropylene, metal alloys and glass.

A pair of poles which include a glass disc mounted on a rod required to attach it via the jaws of the extensometer can be used as supports. The disc can be the size of the pole and attached thereto by means of an adhesive of the Araldite® type. The styling composition to be tested is distributed as uniformly as possible over the surface of each glass disc and dried such that the surface remains flat.

The peel profile is determined by measuring Fmax corresponding to the tensile force, measured using the extensometer, required to peel apart the respective surfaces of the two discs. The process can be performed according to the following protocol: 6 pairs of poles are prepared. For each pair of poles, a peel test is performed according to the procedure indicated above for test 1. The results obtained on the 6 peel profiles performed are selected, excluding for each pair of poles the cases where the styling materials have become peeled from one of the poles of the pair. The Fmax is determined for each remaining peel profile. The average of these measurements is determined.

In one embodiment of the present disclosure, the at least one non-silicone polymer may be chosen so as to give the composition defined above a separation energy Es(M/V) of less than 300 μJ.

According to the present disclosure, the term “Es(M/V)” means the energy supplied by the extensometer to perform the “separation” of the respective 38 mm² surfaces of two rigid, inert, non-absorbent supports (C) and (D) placed facing each other; one of the said supports consisting of polished glass and the other of the said supports being of a nature identical to that of the supports (A) and (B) defined above and whose surface is coated with the composition defined above at a rate of 519 μg/mm² on the support, dried for 24 hours at 22° C. under a relative humidity of 50%, and then subjected for 20 seconds to a compression of 3 newtons and finally subjected for 30 seconds to a traction at a speed of 30 mm/minute.

This energy supplied by the extensometer is the work determined by the integral of F(x)dx between Xs1+0.05 and Xs2; where F(x) is the force required to produce a displacement (x); Xs1 is the displacement (expressed in millimetres) produced by the maximum tensile force; Xs2 is the displacement (expressed in millimetres) produced by the tensile force that allows total separation of the two surfaces.

Procedure relating to the mean of Es(M/V): the energy supplied by the extensometer to perform the “separation” of the respective surfaces of two 38 mm² rigid, inert, non-absorbent supports placed facing each other is determined; one of the said supports consisting of polished glass and the other of the said supports being of a nature identical to that of the supports defined above and whose surface is coated and treated under the same conditions as those of the first procedure described above and using an extensometer of the same type as previously. The process can be performed according to the following protocol: 6 pairs of poles are prepared. A peel test according to the procedure indicated above is performed for each pair of poles. The results obtained on the 6 peel profiles performed are selected, excluding for each pair of poles the cases where the styling materials have become peeled from one of the poles of the pair. The Es(M/V) is determined for each remaining peel profile. The mean of these measurements is determined.

According to an embodiment of the present disclosure, the at least one non-silicone polymer may be dendritic, linear or branched, comb, block or star shaped. It may contain one or more types of repeating unit and may thus be chosen from random, alternating or block homopolymers and copolymers. According to the present disclosure, the term “polymer” means a compound comprising at least 5 repeating units linked together via covalent bonds. In an embodiment of the present disclosure, the at least one non-silicone polymer may be chosen from amphiphilic block linear copolymers.

For the purposes of the present disclosure, the term “non-silicone” refers to a polymer not containing any —SiOSi— bonds.

The main skeleton of the at least one non-silicone polymer may comprise C and H atoms, and may be interrupted with one or more heteroatoms such as O, N, P and S, and may comprise chain-end functions or side functions.

The block copolymers may be linear or star, di-, tri- or multiblock. They may be water-soluble, water-dispersible or liposoluble.

When the polymers dissolve or disperse spontaneously or by neutralization in water, they may include at least one block of hydrophilic nature and of at least one block of hydrophilic nature of different chemical composition from the previous block, or alternatively they may include at least one block of hydrophilic nature and at least one block of hydrophobic nature. The polymers may be of anionic, cationic, nonionic or amphoteric nature.

When the polymers dissolve or disperse spontaneously in an anhydrous medium, they may include at least one block of hydrophobic nature and of at least one block of hydrophobic nature of different chemical composition from the preceding block, or alternatively they may include at least one block of hydrophobic nature and at least one block of hydrophilic nature. The polymers may be of anionic, cationic, nonionic or amphoteric nature.

The terms “water-soluble, water-dispersible or lipodispersible block copolymer” all refer to a copolymer which, at a concentration of 0.1% active material in water or in an anhydrous solvent at 25° C., leads spontaneously, or after neutralization using an acid or a base, to a macroscopically homogeneous, transparent or translucent solution or suspension, i.e., a solution or suspension having a transmittance value at a wavelength of 500 nm through a sample 1 cm thick of at least 70%, such as, for example, 80%.

The term “block of hydrophilic nature” means a block consisting of at least 75% by weight of monomers that are water-soluble and/or water-dissolvable by neutralization. The hydrophilic block may thus contain up to 25 mol %, such as, for example, 10 mol % or less than or equal to 5 mol %, of one or more water-insoluble monomers.

The term “block of hydrophobic nature” means a block consisting of at least 75% by weight of water-insoluble monomers. The hydrophilic block may thus contain up to 25 mol %, such as, for example, 10 mol % or less than or equal to 5 mol %, of one or more water-soluble monomers.

As examples of monomers that may be used in the polymers of the invention, non-limiting mention may be made of those described in French Patent Application No. FR 2 840 205.

In one embodiment, water-soluble monomers forming the hydrophilic block(s) of the diblock copolymers that can be used according to the present disclosure may be of anionic, nonionic or cationic nature and may be used alone or in the form of a mixture containing two or more different monomers.

Non-limiting examples of anionic water-soluble monomers that may be mentioned include ethylenically unsaturated carboxylic acids, such as acrylic acid, methacrylic acid, itaconic acid, fumaric acid, crotonic acid, maleic acid, 2-acrylamido-2-methylpropanesulfonic acid, styrenesulfonic acid, vinylsulfonic acid and vinylphosphonic acid.

The nonionic water-soluble monomers that may be used according to the present disclosure may include acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolyzed), ethylene oxide, hydroxyethyl acrylate, hydroxypropyl acrylate, hydroxyethyl methacrylate and hydroxypropyl methacrylate.

Cationic water-soluble monomers that may be used according to the present disclosure may include, for example, dimethyldiallylammonium chloride, methylvinylimidazolium chloride, 2-vinylpyridine, 4-vinylpyridine, 2-methyl-5-vinylpyridine, N-(C₁₋₄ alkyl)-4-vinylpyridinium halides such as N-methyl-4-vinylpyridinium iodide, vinylamine and the monomers of formula:
H₂C═CR′₁—CO—X₂

wherein:

R′₁ is chosen from a hydrogen atom and a methyl group; and

X₂ is chosen from a linear or branched C₁₋₆ hydrocarbon-based group bearing at least one primary, secondary or tertiary amine function or at least one quaternary nitrogen atom, a group of formula NHR′₂, a group of formula NR′₂R′₃, wherein R′₂ and R′₃ chosen from, independently of each other, a linear or branched C₁₋₆ hydrocarbon-based group bearing at least one primary, secondary or tertiary amine function or at least one quaternary nitrogen atom.

The water-insoluble monomers forming the hydrophobic block(s) of the diblock copolymers that may be used according to the present disclosure can be chosen from vinylaromatic monomers such as styrene and its alkyl derivatives, for instance 4-butylstyrene, α-methylstyrene and vinyltoluene; dienes such as butadiene and 1,3-hexadiene; alkyl derivatives of dienes, such as isoprene and dimethylbutadiene; chloroprene; C₁₋₁₀ alkyl; C₆₋₁₀ aryl acrylates; C₆₋₁₀ aralkyl acrylates; and C₁₋₁₀ alkyl, C₆₋₁₀ aryl or C₆₋₁₀ aralkyl methacrylates, for instance methyl, ethyl, n-butyl, 2-ethylhexyl, tert-butyl, isobornyl, phenyl or benzyl (meth)acrylate, vinyl acetate, the vinyl ethers of formula CH₂═CH—O—R″ and the allyl ethers of formula CH₂═CH—CH₂—O—R″, wherein R″ is chosen from a C₁₋₆ alkyl group, acrylonitrile, vinyl chloride, vinylidene chloride, caprolactone, ethylene, propylene, vinyl monomers that are fluorinated or that contain a perfluoro chain, such as fluoroalkyl acrylates or methacrylates, and alkyl α-fluoroalkylates.

In one embodiment, the non-silicone polymer according to the present disclosure may be chosen from non-silicone fixing polymers.

In an embodiment of the present disclosure, the non-silicone fixing polymers may be chosen from cationic, anionic, amphoteric and nonionic non-silicone fixing polymers, and mixtures thereof.

In one embodiment, the cationic non-silicone fixing polymers that may be used according to the present disclosure may be chosen from polymers comprising primary, secondary, tertiary and/or quaternary amine groups forming part of the polymer chain or directly attached thereto, and having a number average molecular weight ranging from 500 to 5,000,000, such as, for example, from 1,000 to 3,000,000.

Among the non-silicone polymers that may be used according to an embodiment of the present disclosure, non-limiting mention may be made of the following cationic polymers:

(1) homopolymers or copolymers derived from acrylic or methacrylic esters or amides with amine functions, and comprising at least one of the units of the following formulae: 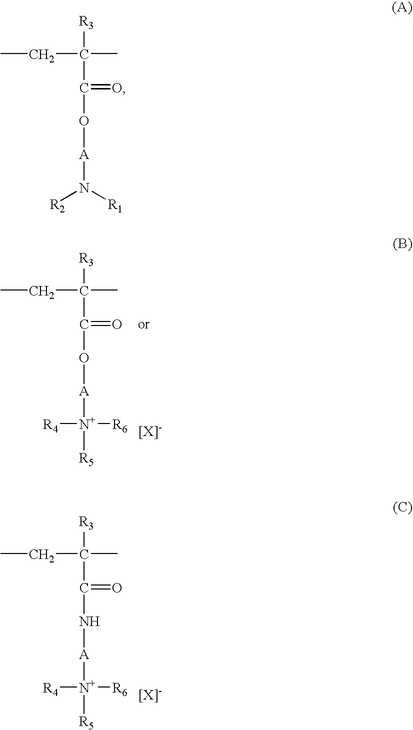
wherein:

R₁ and R₂, which may be identical or different, are chosen from a hydrogen atom and alkyl groups having from 1 to 6 carbon atoms;

R₃ is chosen from a hydrogen atom and a group CH₃;

A is chosen from linear or branched alkyl groups comprising 1 to 6 carbon atoms and hydroxyalkyl groups comprising 1 to 4 carbon atoms;

R₄, R₅ and R₆, which may be identical or different, are chosen from alkyl groups having from 1 to 18 carbon atoms and a benzyl group;

X is chosen from a methosulfate anion and a halide such as, for example, chloride or bromide.

The copolymers of the family (1) may also contain one or more units derived from comonomers that may be chosen from the family of acrylamides, methacrylamides, diacetone acrylamides, acrylamides and methacrylamides substituted on the nitrogen with lower (C₁₋₄) alkyl groups, groups derived from acrylic or methacrylic acids or esters thereof, vinyllactams such as vinylpyrrolidone or vinylcaprolactam, and vinyl esters.

Thus, among these additional copolymers of the family (1), non-limiting mention may be made of:
copolymers of acrylamide and of dimethylaminoethyl methacrylate quaternized with dimethyl sulfate or with a dimethyl halide, such as the one sold under the name Hercofloc® by the company Hercules;
copolymers of acrylamide and of methacryloyloxyethyltrimethylammonium chloride, described, for example, in European Patent Application No. EP-A-080 976 and sold under the name Bina Quat P 100 by the company Ciba Geigy;
copolymers of acrylamide and of methacryloyloxyethyltrimethylammonium methosulfate, such as the product sold under the name Reten by the company Hercules;
quaternized or non-quaternized vinylpyrrolidone/dialkylaminoalkyl acrylate or methacrylate copolymers, such as the products sold under the name “Gafquat®” by the company ISP, such as, for example, “Gafquat® 734” or “Gafquat®755”, or alternatively the products known as “Copolymer® 845, 958 and 937”. These polymers are described in detail in French Patent Nos. 2 077 143 and 2 393 573;
fatty-chain polymers containing a vinylpyrrolidone unit, such as the products sold under the name Styleze W20 and Styleze W10 by the company ISP;
dimethylaminoethyl methacrylate/vinylcaprolactam/vinylpyrrolidone terpolymers, such as the product sold under the name Gaffix® VC 713 by the company ISP; and
quaternized vinylpyrrolidone/dimethylaminopropylmethacrylamide copolymers, such as the product sold under the name “Gafquat® HS 100” by the company ISP;

(2) cationic polysaccharides, such as, for example, those containing quaternary ammonium, such as those described in U.S. Pat. Nos. 3,589,578 and 4,031,307, such as guar gums containing trialkylammonium cationic groups. Such products include, for example, those sold under the trade names Jaguar C13S, Jaguar C15 and Jaguar C17 by the company Meyhall;

(3) quaternary copolymers of vinylpyrrolidone and of vinylimidazole;

(4) chitosans or salts thereof; the salts that can be used, include, for example, chitosan acetate, lactate, glutamate, gluconate or pyrrolidonecarboxylate.

Among the compounds of family (4) that may be used according to the present disclosure, non-limiting mention may be made of chitosan having a degree of deacetylation of 90.5% by weight, sold under the name Kytan Brut Standard by the company Aber Technologies, and chitosan pyrrolidonecarboxylate sold under the name Kytamer® PC by the company Amerchol.

(5) cationic cellulose derivatives such as copolymers of cellulose or of cellulose derivatives grafted with a water-soluble monomer comprising a quaternary ammonium, and disclosed, for example, in U.S. Pat. No. 4,131,576, such as hydroxyalkylcelluloses, for instance hydroxymethyl-, hydroxyethyl- or hydroxypropylcelluloses grafted with a methacryloyloxyethyltrimethylammonium, methacrylamidopropyltrimethylammonium or dimethyidiallylammonium salt.

The products sold corresponding to family (5), may include, for example, the products sold under the name “Celquat L 200” and “Celquat H 100” by the company National Starch.

Anionic non-silicone fixing polymers that may be used according to the present disclosure, may include polymers comprising groups derived from carboxylic acid, sulfonic acid or phosphoric acid and have a number average molecular weight ranging from 500 to 5,000,000.

According to one embodiment, the carboxylic groups that may be used according to the present disclosure may be provided by unsaturated monocarboxylic or dicarboxylic acid monomers such as those corresponding to the formula (i):

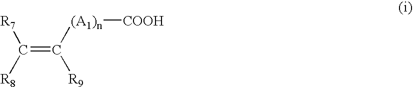

wherein:

n is an integer ranging from 0 to 10;

A₁ is a methylene group, optionally connected to the carbon atom of the unsaturated group, or to the neighbouring methylene group when n is greater than 1, via a hetero atom such as, for example, oxygen or sulfur;

R₇ is chosen from a hydrogen atom, a phenyl, and a benzyl group;

R₈ is chosen from a hydrogen atom, a lower alkyl, and a carboxyl group;

R₉ is chosen from a hydrogen atom, a lower alkyl group, a —CH₂—COOH, phenyl and a benzyl group.

In the abovementioned formula, a lower alkyl group means a group having 1 to 4 carbon atoms, such as, for example, methyl and ethyl groups.

The anionic non-silicone fixing polymers containing carboxylic groups that may be used according to the present disclosure may include:

A) acrylic or methacrylic acid homo- or copolymers, or salts thereof, such as the products sold under the names Versicol® E or K by the company Allied Colloid and Ultrahold® by the company BASF, the copolymers of acrylic acid and of acrylamide sold in the form of their sodium salts under the names Reten 421, 423 or 425 by the company Hercules, and the sodium salts of polyhydroxycarboxylic acids.

B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described, for example, in French Patent No.1 222 944 and German Patent Application No. 2 330 956, the copolymers of this type comprising an optionally N-alkylated and/or hydroxyalkylated acrylamide unit in their chain as described, for example, in Luxembourg Patent Application Nos. 75370 and 75371, or sold under the name Quadramer by the company American Cyanamid. Non-limiting mention may also be made of copolymers of acrylic acid and of C₁-C₄ alkyl methacrylate; and, terpolymers of vinylpyrrolidone, of acrylic acid and of methacrylate of C₁-C₂₀ alkyl, for example of lauryl, such as the product sold by the company ISP under the name Acrylidone® LM and methacrylic acid/ethyl acrylate/tert-butyl acrylate terpolymers such as the product sold under the name Luvimer® 100 P by the company BASF. Non-limiting mention may also be made of methacrylic acid/acrylic acid/ethyl acrylate/methyl methacrylate copolymers as an aqueous dispersion, sold under the name Amerhold® DR 25 by the company Amerchol.

C) crotonic acid copolymers, such as those comprising vinyl acetate or propionate units in their chain, and optionally other monomers such as allylic esters or methallylic esters, vinyl ether or vinyl ester of a linear or branched saturated carboxylic acid with a long hydrocarbon chain such as, for example, those containing at least 5 carbon atoms, it being possible for these polymers optionally to be grafted or crosslinked, or alternatively another vinyl, allylic or methallylic ester monomer of an α- or β-cyclic carboxylic acid. Such polymers are described, for example, in French Patent Nos. 1 222 944, 1 580 545, 2 265 782, 2 265 781, 1 564 110 and 2 439 798. Commercial products falling into this class include the resins 28-29-30, 26-13-14 and 28-13-10 sold by the company National Starch.

D) copolymers of C₄-C₈ monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described, for example, in U.S. Pat. Nos. 2,047,398; 2,723,248; and 2,102,113; and in British Patent No. GB 839 805, and may also include those sold under the names Gantrez® AN or ES by the company ISP;
copolymers comprising (i) one or more maleic, citraconic or itaconic anhydride units and (ii) one or more monomers chosen from allylic or methallylic esters optionally comprising one or more acrylamide, methacrylamide, α-olefin, acrylic or methacrylic esters, acrylic or methacrylic acid or vinylpyrrolidone groups in their chain, the anhydride functions of these copolymers optionally being monoesterified or monoamidated. These polymers are described, for example, in French Patent Nos. FR 2 350 384 and FR 2 357 241 by the Applicant; and

E) polyacrylamides comprising carboxylate groups.

The homopolymers and copolymers comprising sulfonic groups may include polymers comprising vinylsulfonic, styrenesulfonic, naphthalenesulfonic or acrylamidoalkyl-sulfonic units.

Among these polymers that may be used according to the present disclosure, non-limiting mention may be made of:
polyvinylsulfonic acid salts having a molecular weight approximately ranging from 1000 to 100,000, as well as the copolymers with an unsaturated comonomer, such as acrylic or methacrylic acids and their esters, as well as acrylamide or its derivatives, vinyl ethers and vinylpyrrolidone;
polystyrenesulfonic acid salts, such as the sodium salts, that are sold under the names Flexan® 500 and Flexan® 130 by National Starch. These compounds are described in French Patent No. FR 2 198 719;
polyacrylamidesulfonic acid salts, such as those mentioned in U.S. Pat. No. 4,128,631, and also polyacrylamideethylpropanesulfonic acid sold under the name Cosmedia Polymer HSP 1180 by Henkel.

As another anionic non-silicone fixing polymer that can be used according to the present disclosure, non-limiting mention may be made of the branched block anionic polymers sold under the name Fixate G100 by the company Noveon.

According to one embodiment of the present disclosure, the anionic non-silicone fixing polymers may be chosen from acrylic acid copolymers, such as the acrylic acid/ethyl acrylate/N-tert-butylacrylamide terpolymers sold, for example, under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold, for example, under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold under the name Gantrez® [lacuna] by the company ISP, the copolymers of methacrylic acid and of methyl methacrylate sold under the name Eudragit® L by the company Rohm Pharma, the copolymers of methacrylic acid and of ethyl acrylate sold under the name Luvimer® MAEX or MAE by the company BASF, the vinyl acetate/crotonic acid copolymers sold under the name Luviset CA 66 by the company BASF, the vinyl acetate/crotonic acid copolymers grafted with polyethylene glycol sold under the name Aristoflex A® by the company BASF, and the polymer sold under the name Fixate G100 by the company Noveon.

Among the anionic non-silicone fixing polymers mentioned above, a further embodiment of the present disclosure may use the methyl vinyl ether/monoesterified maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer® MAEX or MAE by the company BASF, the vinylpyrrolidone/acrylic acid/lauryl methacrylate terpolymers sold under the name Acrylidone® LM by the company ISP, and the polymer sold under the name Fixate G100 by the company Noveon.

Among the amphoteric non-silicone fixing polymers that can be used in accordance with the present disclosure, non-limiting mention can be made of polymers comprising units B and C distributed randomly in the polymer chain, wherein B is chosen from at least one unit derived from a monomer comprising at least one basic nitrogen atom and C is chosen from at least one unit derived from an acid monomer comprising one or more carboxylic or sulfonic groups, or alternatively B and C may be chosen from groups derived from carboxybetaine and sulfobetaine zwitterionic monomers.

B and C may also be chosen from a cationic polymer chain comprising primary, secondary, tertiary or quaternary amine groups, in which at least one of the amine groups bears a carboxylic or sulfonic group connected via a hydrocarbon group, or alternatively B and C form part of a chain of a polymer containing an α, β-dicarboxylic ethylene unit in which one of the carboxylic groups has been made to react with a polyamine comprising one or more primary or secondary amine groups.

In one embodiment, the amphoteric non-silicone fixing polymers that may be used according to the present disclosure, may be chosen from the following polymers:

(1) copolymers having acidic vinyl and basic vinyl units, such as those resulting from the copolymerization of a monomer derived from a vinyl compound bearing a carboxylic group such as, for example, acrylic acid, methacrylic acid, maleic acid, α-chloroacrylic acid, and a basic monomer derived from a substituted vinyl compound containing at least one basic atom, such as, for example, dialkylaminoalkyl methacrylate and acrylate, dialkylaminoalkylmethacrylamides and -acrylamides. Such compounds are described in U.S. Pat. No. 3,836,537.

(2) polymers comprising units derived from:
a) at least one monomer chosen from acrylamides and methacrylamides substituted on the nitrogen atom with an alkyl group,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that can be used according to an embodiment of the present disclosure include compounds in which the alkyl groups contain from 2 to 12 carbon atoms, such as, for example, N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers that may be used according to a further embodiment of the present disclosure may be chosen, for example, from acrylic acid, methacrylic acid, crotonic acid, itaconic acid, maleic acid and fumaric acid and alkyl monoesters, having 1 to 4 carbon atoms, of maleic or fumaric acids or anhydrides.

In one embodiment, the comonomers may be chosen from aminoethyl, butylaminoethyl, N,N′-dimethylaminoethyl and N-tert-butylaminoethyl methacrylates.

The copolymers whose CTFA (4th edition, 1991) name is octylacryl-amide/acrylates/butylaminoethyl methacrylate copolymer, such as the products sold under the name Amphomer® or Lovocryl® 47 by the company National Starch, may also be used in a composition according to the present disclosure.

(3) crosslinked and acylated polyamino amides partially or totally derived from polyamino amides of general formula (ii): [symbol]
CO—R₁₀—CO-Z [symbol] (ii)

wherein:

R₁₀ is chosen from a divalent group derived from a saturated dicarboxylic acid, a mono- or dicarboxylic aliphatic acid containing an ethylenic double bond, an ester of a lower alkanol, having 1 to 6 carbon atoms, of these acids, and a group derived from the addition of any one of said acids to a bis(primary) or bis(secondary) amine; and

Z is chosen from a group derived from a bis(primary), mono- or bis(secondary) polyalkylene-polyamine and may represent:

a) in proportions of from 60 to 100 mol %, the group (iii):
—NH [symbol] (CH₂)ₓ—NH [symbol] ₚ (iii)

where x=2 and p=2 or 3, or alternatively x=3 and p=2

this group being derived from diethylenetriamine, from triethylenetetraamine or from dipropylenetriamine;

b) in proportions of from 0 to 40 mol %, the group (iii) above in which x=2 and p=1 and which is derived from ethylenediamine, or the group derived from piperazine:

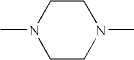

c) in proportions of from 0 to 20 mol %, the —NH(CH₂)₆—NH— group being derived from hexamethylenediamine,

these polyamino amides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the acylation may include propane sultone or butane sultone, the salts of the acylating agents may include the sodium or potassium salts.

(4) polymers comprising zwitterionic units of formula (iv): 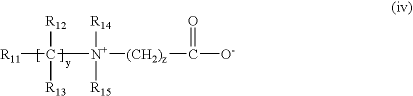
wherein:

R₁₁ is chosen from a polymerizable unsaturated group such as an acrylate, methacrylate, acrylamide or methacrylamide group;

y and z are chosen from an integer ranging from 1 to 3;

R₁₂ and R₁₃ are chosen from a hydrogen atom, a methyl, a ethyl and a propyl group; and

R₁₄ and R₁₅ are chosen from a hydrogen atom and an alkyl group such that the sum of the carbon atoms in R₁₄ and R₁₅ does not exceed 10.

The polymers comprising such units can also comprise units derived from nonzwitterionic monomers such as dimethyl- or diethylaminoethyl acrylate or methacrylate or alkyl acrylates or methacrylates, acrylamides or methacrylamides or vinyl acetate.

By way of example, non-limiting mention may be made of the copolymers of methyl methacrylate/methyl dimethylcarboxymethylammonioethyl methacrylate such as the product sold under the name Diaformer Z301 by the company Sandoz.

(5) polymers derived from chitosan comprising monomer units corresponding to the following formulae:

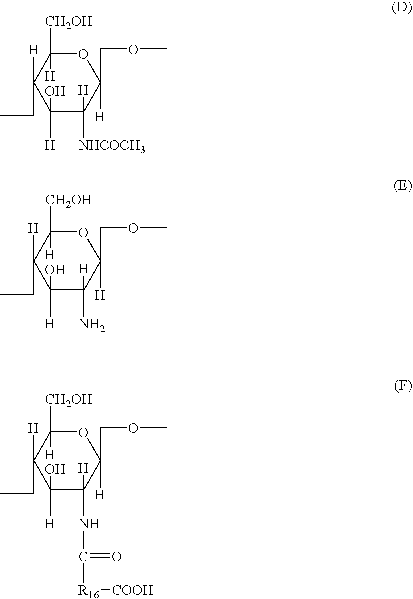

wherein the unit (D) is present in proportions ranging from 0 and 30%, the unit (E) in proportions ranging from 5% to 50% and the unit (F) in proportions ranging from 30% to 90%, wherein in unit (F), R₁₆ is chosen from a group of formula:

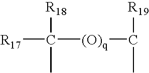

wherein:

if q=0, R₁₇, R₁₈ and R₁₉, which may be identical or different, are chosen from a hydrogen atom; a methyl; hydroxyl; acetoxy residue; amino residue; a monoalkylamine residue or a dialkylamine residue, optionally interrupted by at least one nitrogen atoms and/or optionally substituted with at least one amine, hydroxyl, carboxyl, alkylthio or sulfonic groups; and an alkylthio residue wherein the alkyl group bears an amino residue; wherein at least one of the groups R₁₇, R₁₈ and R₁₉ chosen from a hydrogen atom;

or, if q=1, R₁₇, R₁₈ and R₁₉ are chosen from a hydrogen atom;

as well as the salts formed by these compounds with bases or acids.

(6) polymers corresponding to the general formula (v) that are described, for example, in French Patent No. 1 400 366:

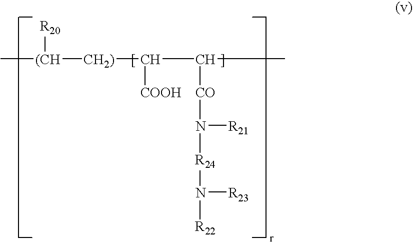

wherein:

R₂₀ is chosen from a hydrogen atom, a CH₃O, CH₃CH₂O and a phenyl group;

R₂₁ is chosen from a hydrogen atom and a lower alkyl group such as, for example, methyl or ethyl;

R₂₂ is chosen from a hydrogen atom and a C₁₋₆ lower alkyl group such as, for example, methyl or ethyl;

R₂₃ is chosen from a C₁₋₆ lower alkyl group such as, for example, methyl or ethyl, and a group corresponding to the formula: —R₂₄—N(R₂₂)₂,

wherein R₂₄ is chosen from a —CH₂—CH₂—, —CH₂—CH₂—CH₂— and —CH₂—CH(CH₃)— group, and R₂₂ having the meanings mentioned above;

(7) polymers derived from the N-carboxyalkylation of chitosan, such as, for example, N-carboxymethylchitosan or N-carboxybutylchitosan sold under the name “Evalsan” by the company Jan Dekker.

(8) amphoteric polymers of the type -D-X-D-X- chosen from:

a) polymers obtained by the action of chloroacetic acid or sodium chloroacetate on compounds comprising at least one unit of formula (vi):
-D-X-D-X-D- (vi)

wherein D is

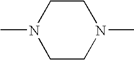

and X is chosen from E and E′,

wherein E and E′, which may be identical or different, are chosen from a divalent group that is an alkylene group with a straight or branched chain containing up to 7 carbon atoms in the main chain, which is unsubstituted or substituted with hydroxyl groups and which can comprise, in addition to the oxygen, nitrogen and sulfur atoms, 1 to 3 aromatic and/or heterocyclic rings;

wherein the oxygen, nitrogen and sulfur atoms are present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;

b) polymers of formula (vi′):
-D-X-D-X- (vi′)

where D is

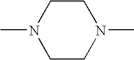

and X is chosen from E and E′,

wherein X comprises at least one E′;

wherein E is defined as above; and

E′ is chosen from a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

Among the amphoteric non-silicone fixing polymers described above, one embodiment according to the present disclosure comprises those of family (3), such as, for example, the copolymers whose CTFA name is octylacrylamide/acrylates/butylaminoethyl methacrylate copolymer, such as the products sold under the names Amphomer®, Amphomer® LV 71 or Lovocryl® 47 by the company National Starch and those of family (4) such as the copolymers of methyl methacrylate/methyl dimethylcarboxymethylammonio-ethyl methacrylate, sold, for example, under the name Diaformer® Z301 by the company Sandoz.

In one embodiment, the nonionic non-silicone fixing polymers that may be used according to the present disclosure are chosen, for example, from:
polyalkyloxazolines;
vinyl acetate homopolymers;
vinyl acetate copolymers, for instance copolymers of vinyl acetate and of acrylic ester; copolymers of vinyl acetate and of ethylene; copolymers of vinyl acetate and of maleic ester, for example of dibutyl maleate;
homopolymers and copolymers of acrylic esters, for instance copolymers of alkyl acrylates and of alkyl methacrylates, such as the products sold by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl (meth)acrylates; non-limiting mention may be made of the products sold under the name CJ 0601 B by the company Rohm & Haas;
styrene homopolymers;
styrene copolymers, for instance copolymers of styrene and of an alkyl (meth)acrylate, such as the products Mowilith® LDM 6911, Mowilith® DM 611 and Mowilith® LDM 6070 sold by the company Hoechst, and the products Rhodopas® SD 215 and Rhodopas® DS 910 sold by the company Rhône-Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers other than vinylpyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF; and poly(vinylpyrrolidone/vinyl acetate/vinyl propionate) terpolymers, for instance the product sold under the name Luviskol® VAP 343 by the company BASF.

In a further embodiment, the alkyl groups of the nonionic polymers mentioned above may contain from 1 to 6 carbon atoms.

Functionalized or non-functionalized, cationic, nonionic, anionic or amphoteric polyurethanes or mixtures thereof may also be used as non-silicone fixing polymers in embodiments according to the present disclosure.

Among the polyurethanes that may be used according to the present disclosure, non-limiting mention may be made of those described in patent applictions EP 0 751 162, EP 0 637 600, EP 0 648 485 and FR 2 743 297, of which the Applicant is the proprietor, and also of patent applications EP 0 656 021 and WO 94/03510 from the company BASF, and EP 0 619 111 from the company National Starch.

As polyurethanes that may be used according to the present disclosure, non-limiting mention may be made of the product sold under the name Luviset Pur® by the company BASF.

In one embodiment of the present disclosure, the at least one non-silicone polymer may be present in the composition in an amount ranging from 0.05% to 99% by weight, such as, for example, from 0.1% to 95% or from 0.2% to 30% by weight, relative to the total weight of the composition.

The term “electrophilic monomer” means a monomer capable of polymerizing via anionic polymerization in the presence of a nucleophilic agent, such as, for example, the hydroxyl ions (OH—) contained in water.

The term “anionic polymerization” means the mechanism defined in the book “Advanced Organic Chemistry”, Third Edition, by Jerry March, pages 151 to 161.

According to an embodiment of the present disclosure, the at least one electrophilic monomer present in the composition may be chosen from:
the benzylidene malononitrile derivatives (A), 2-(4-chloro-benzylidene)malononitrile (A1), ethyl 2-cyano-3-phenylacrylate (B), and ethyl 2-cyano-3-(4-chlorophenyl)acrylate (B1) described in Sayyah, J. Polymer Research, 2000, p. 97, 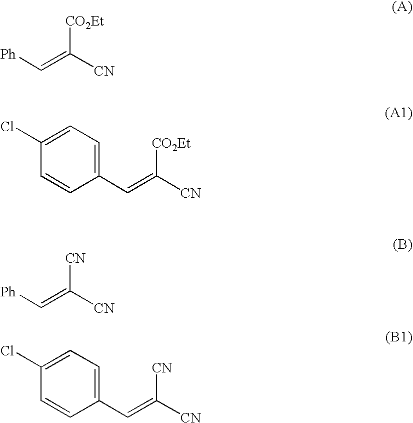
methylidenemalonate derivatives, such as:
diethyl 2-methylenemalonate (C) described in Hopff, Makromoleculare Chemie [Macromolecular Chemistry], 1961, p. 95, De Keyser, J. Pharm. Sci, 1991, p. 67 and Klemarczyk, Polymer, 1998, p.173, 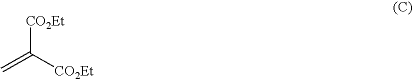
ethyl 2-ethoxycarbonylmethylenecarbonylacrylate (D) described in Breton, Biomaterials, 1998, p. 271 and Couvreur, Pharmaceutical Research, 1994, p.1270, 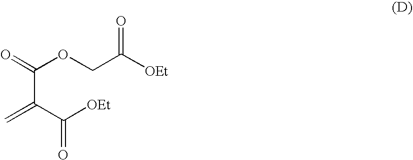
itaconate and itaconimide derivatives, such as:
dimethyl itaconate (E) described in Bachrach, European Polymer Journal, 1976, p.563, 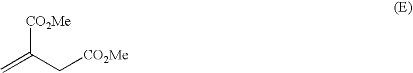
N-butyl itaconimide (F), N-(4-tolyl) itaconimide (G), N-(2-ethylphenyl)itaconimide (H), N-(2,6-diethylphenyl) itaconimide (I) described in Wanatabe, J. Polymer Scienc: Part A: Polymer chemistry, 1994, p. 2073, 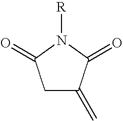
   Bu (F), 4-tolyl (G), 2-ethylphenyl (H), 2,6-diethyphenyl (I)
the derivatives methyl α-(methylsulfonyl)acrylate (K), ethyl α-(methylsulfonyl)acrylate (L), methyl α-(tert-butylsulfonyl)acrylate (M), tert-butyl α-(methylsulfonyl)acrylate (N) and tert-butyl α-(tert-butylsulfonyl)acrylate (O), described in Gipstein, J. Org. Chem, 1980, p. 1486, 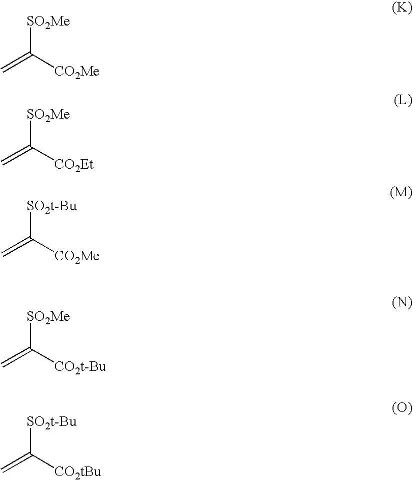
the derivatives 1,1-bis(methylsulfonyl)ethylene (P), 1-acetyl-1-methylsulfonylethylene (Q), methyl α-(methylsulfonyl)vinylsulfonate (R) and α-methylsulfonylacrylonitrile (S) described in Shearer, U.S. Pat. No. 2,748,050, 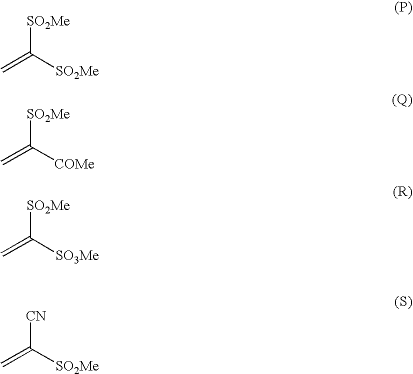
the methyl vinyl sulfone (T) and phenyl vinyl sulfone (U) derivatives described in Boor, J. Polymer Science, 1971, p. 249, 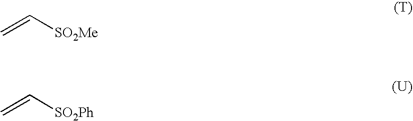
the phenyl vinyl sulfoxide derivative (V) described in Kanga, Polymer preprints (ACS, Divison of Polymer Chemistry), 1987, p. 322, 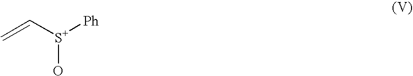
the derivative 3-methyl-N-(phenylsulfonyl)-1 -aza-1,3-butadiene (W) described in Bonner, Polymer Bulletin, 1992, p. 517, 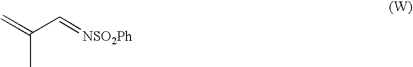
acrylate and acrylamide derivatives, for instance:
N-propyl-N-(3-triisopropoxysilylpropyl)acrylamide (X) and N-propyl-N-(3-triethoxysilylpropyl)acrylamide (Y) described by Kobayashi, Journal of Polymer Science, Part A: Polymer Chemistry, 2005, p. 2754, 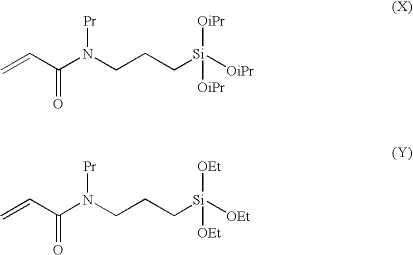
2-hydroxyethyl acrylate (Z) and 2-hydroxyethyl methacrylate (AA) described in Rozenberg, International Journal of Plastics Technology, 2003, p. 17, 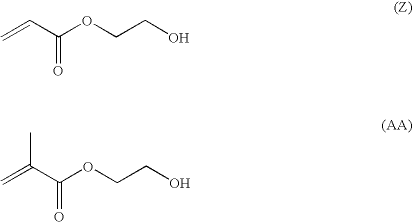
N-butyl acrylate (AB) by Schmitt, Macromolecules, 2001, p. 2115, 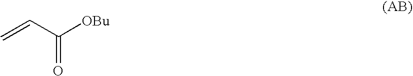
Tert-butyl acrylate (AC) by Ishizone, Macromolecules, 1999, p. 955.

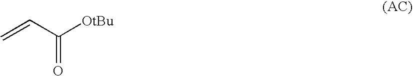

An electron-withdrawing monomer that may be used according to an embodiment of the present disclosure may be cyclic or linear. When it is cyclic, the electron-withdrawing group is may be exocyclic, i.e., it does not form an integral part of the cyclic structure of the monomer.

According to one embodiment, the electron-withdrawing monomers contain at least two electron-withdrawing groups.

As examples of monomers containing at least two electron-withdrawing groups that may be used according to the present disclosure, non-limiting mention may be made of the monomers of formula (I):

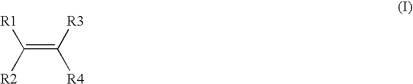

wherein:

R₁ and R₂ are chosen from, independently of each other, a sparingly or non-electron-withdrawing group (sparingly or non-inductive-withdrawing) such as, for example:
a hydrogen atom;
a saturated or unsaturated, linear, branched or cyclic hydrocarbon-based group preferably containing from 1 to 20 carbon atoms, such as from 1 to 10 carbon atoms, and optionally containing at least one nitrogen, oxygen or sulfur atom, and optionally substituted with at least one group chosen from —OR, —COOR, —COR, —SH, —SR and —OH, and halogen atoms;
a modified or unmodified polyorganosiloxane residue;
a polyoxyalkylene group;

R₃ and R₄ comprise, independently of each other, an electron-withdrawing (or inductive-withdrawing) group chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups, linear or branched alkenyl groups, linear or branched alkynyl groups, C₁-C₄ mono- or polyfluoroalkyl groups, aryl groups such as phenyl, or aryloxy groups such as phenoxyloxy;

R is chosen from a saturated or unsaturated, linear, branched or cyclic hydrocarbon-based group, such as, for example, a group containing from 1 to 20 or from 1 to 10 carbon atoms, and optionally containing at least one nitrogen, oxygen or sulfur atom, and optionally substituted with at least one group chosen from —OR′, —COOR′, —COR′, —SH, —SR′ and —OH, halogen atoms, or a polymer residue obtained by free-radical polymerization, by polycondensation or by ring opening;

R′ is chosen from a C₁-C₁₀ alkyl radical.

The term “electron-withdrawing or inductive-withdrawing group (—I)” means any group that is more electronegative than carbon. Reference may be made to the publication P. R. Wells, Prog. Phys. Org. Chem., Vol 6, 111 (1968).

The term “sparingly or non-electron-withdrawing group” means any group whose electronegativity is less than or equal to that of carbon.

The alkenyl or alkynyl groups that can be used according to an embodiment of the present disclosure may contain from 2 to 20 carbon atoms, such as, for example, from 2 to 10 carbon atoms.

As saturated or unsaturated, linear, branched or cyclic hydrocarbon-based groups containing from 1 to 20 carbon atoms or from 1 to 10 carbon atoms that can be used according to the present disclosure, non-limiting mention may be made of linear or branched alkyl, alkenyl or alkynyl groups, such as methyl, ethyl, n-butyl, tert-butyl, isobutyl, pentyl, hexyl, octyl, butenyl or butynyl; cycloalkyl or aromatic groups.

Among examples of substituted hydrocarbon-based groups that may be used according to the present disclosure, non-limiting mention may be made of hydroxyalkyl and polyhaloalkyl groups.

Examples of unmodified polyorganosiloxanes that may be used according to an embodiment of the present disclosure include polyalkylsiloxanes such as polydimethylsiloxanes, polyarylsiloxanes such as polyphenylsiloxanes, and polyarylalkylsiloxanes such as polymethylphenylsiloxanes.

Among the modified polyorganosiloxanes that may be used according to the present disclosure, non-limiting mention may be made of polydimethylsiloxanes containing polyoxyalkylene and/or siloxy and/or silanol and/or amine and/or imine and/or fluoroalkyl groups.

Among the polyoxyalkylene groups that may be mentioned with regard to an embodiment of the disclosure, are polyoxyethylene groups and polyoxypropylene groups containing 1 to 200 oxyalkylene units.

Among the mono- or polyfluoroalkyl groups that may be used according to an embodiment of the present disclosure, non-limiting mention may be made of groups such as —(CH₂)n-(CF₂)m-CF₃ or —(CH₂)n-(CF₂)m-CHF₂ with n=1 to 20 and m=1 to 20.

In a further embodiment of the present disclosure, the substituents R₁ to R₄ may optionally be substituted with a group having cosmetic activity. The cosmetic activities may be obtained from groups having colouring, antioxidant, UV-screening and conditioning functions.

As examples of groups having a colouring function, non-limiting mention may be made of azo, quinone, methine, cyanomethine and triarylmethane groups.

As examples of groups having an antioxidant function, non-limiting mention may be made of groups of butylhydroxyanisole (BHA), butylhydroxytoluene (BHT) or vitamin E type.

As examples of groups having a UV-screening function, non-limiting mention may be made of groups of the benzophenone, cinnamate, benzoate, benzylidenecamphor and dibenzoylmethane type.

As examples of groups having a conditioning function, non-limiting mention may be made especially of cationic groups and groups of fatty ester type.

Among the monomers mentioned above, one embodiment of the present disclosure comprises monomers of the cyanoacrylate family and the derivatives thereof of formula (II):

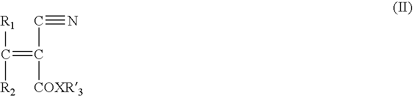

X is chosen from NH, S and O,
R₁ and R₂ are chosen from, independently of each other, a sparingly or non-electron-withdrawing group (sparingly or non-inductive-withdrawing) such as, for example:
a hydrogen atom;
a saturated or unsaturated, linear, branched or cyclic hydrocarbon-based group preferably containing from 1 to 20 carbon atoms, such as from 1 to 10 carbon atoms, and optionally containing at least one nitrogen, oxygen or sulfur atom, and optionally substituted with at least one group chosen from —OR, —COOR, —COR, —SH, —SR and —OH, and halogen atoms;
a modified or unmodified polyorganosiloxane residue;
a polyoxyalkylene group;

R′₃ may be chosen from a hydrogen atom and a radical R,

wherein R is chosen from a saturated or unsaturated, linear, branched or cyclic hydrocarbon-based group, such as, for example, a group containing from 1 to 20 or from 1 to 10 carbon atoms, and optionally containing at least one nitrogen, oxygen or sulfur atom, and optionally substituted with at least one group chosen from —OR′, —COOR′, —COR′, —SH, —SR′ and —OH, halogen atoms, and a polymer residue obtained by free-radical polymerization, by polycondensation or by ring opening.

In one embodiment, X is O.

Non-limiting examples of compounds of formula (II) that may be mentioned include the monomers:

a) belonging to the family of C₁-C₂₀ polyfluoroalkyl 2-cyanoacrylates such as:

the ester 2,2,3,3-tetrafluoropropyl 2-cyano-2-propenoate of formula:

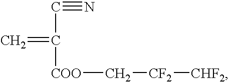

or the ester 2,2,2-trifluoroethyl 2-cyano-2-propenoate of formula:

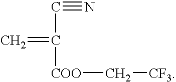

b) the C₁-C₁₀ alkyl or (C₁-C₄ alkoxy)(C₁-C₁₀ alkyl) cyanoacrylates.

Non-limiting mention may also be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

In one embodiment of the present disclosure, the at least one electrophilic monomer is chosen from the monomers b).

In another embodiment, the at least one electrophilic monomer may be chosen from those of formula (III) and mixtures thereof: 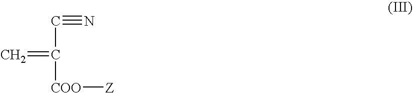
wherein Z is chosen from:
—(CH₂)₇—CH₃;
—CH(CH₃)—(CH₂)₅—CH₃;
—CH₂—CH(C₂H₅)—(CH₂)₃—CH₃;
—(CH₂)₅—CH(CH₃)—CH₃; and
—(CH₂)₄—CH(C₂H₅)—CH₃.

In an embodiment, the at least one electrophilic monomer used in accordance with the present disclosure may be covalently bonded to supports such as, for example, polymers, oligomers or dendrimers. The polymer or the oligomer may be linear, branched, in comb form or in block form. In a further embodiment, the distribution of the at least one electrophilic monomer over the polymeric, oligomeric or dendritic structure may be random, in an end position or in the form of blocks.

The term “cosmetically acceptable medium” means a medium that is compatible with keratin materials such as the hair.

The cosmetically acceptable medium in one embodiment may be anhydrous.

The term “anhydrous medium” means a medium containing less than 1% by weight of water relative to the total weight of the composition.

In one embodiment, the cosmetically acceptable medium may be chosen from organic oils; silicones such as volatile silicones, amino or non-amino silicone gums or oils and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, camelina oil, tamanu oil and lemon oil; waxes; or alternatively organic compounds such as C₅-C₁₀ alkanes, acetone, methyl ethyl ketone, esters of C₁-C₂₀ acids and of C₁-C₈ alcohols such as methyl acetate, butyl acetate, ethyl acetate and isopropylmyristate, dimethoxyethane, diethoxyethane, C₁₀-C₃₀ fatty alcohols such as lauryl alcohol, cetyl alcohol, stearyl alcohol and behenyl alcohol; C₁₀-C₃₀ fatty acids such as lauric acid and stearic acid; C₁₀-C₃₀ fatty amides such as lauric diethanolamide, and C₁₀-C₃₀ fatty alkyl esters such as C₁₀-C₃₀ fatty alkyl benzoates, and mixtures thereof.

In an embodiment comprising organic compounds, the organic compounds may be chosen from compounds that are liquid at a temperature of 25° C. and at 105 Pa (760 mm Hg).

In one embodiment of the compositions used in accordance with the present disclosure, the compositions may have a concentration of the at least one electrophilic monomer ranging from 0.001% to 80% by weight, such as, for example, from 0.1% to 40% by weight or from 1% to 20% by weight, relative to the total weight of the composition.

In other embodiments of the present disclosure, polymerization inhibitors, such as, for example, anionic and/or free-radical polymerization inhibitors may also be introduced into the compositions, in order to enhance the stability of the composition over time. In a non-limiting manner, the following polymerization inhibitors may be mentioned: sulfur dioxide, nitric oxide, lactone, boron trifluoride, hydroquinone and derivatives thereof such as hydroquinone monoethyl ether, tert-butylhydroquinone (TBHQ), benzoquinone and derivatives thereof such as duroquinone, catechol and derivatives thereof such as t-butylcatechol and methoxycatechol, anisole and derivatives thereof such as methoxyanisole, hydroxyanisole or butylhydroxyanisole, pyrogallol, 2,4-dinitrophenol, 2,4,6-trihydroxybenzene, p-methoxyphenol, hydroxybutyltoluene, alkyl sulfates, alkyl sulfites, alkyl sulfones, alkyl sulfoxides, alkyl sulfides, mercaptans and 3-sulfonene, and mixtures thereof. In one embodiment, the alkyl groups may be chosen from groups containing 1 to 6 carbon atoms.

In other embodiments, mineral or organic acids may be used, the latter containing one or more carboxylic or sulfonic groups, with a pKa of between 0 and 6, such as phosphoric acid, hydrochloric acid, nitric acid, benzenesulfonic acid, toluenesulfonic acid, sulfuric acid, carbonic acid, hydrofluoric acid, acetic acid, formic acid, propionic acid, benzoic acid, mono-, di- or trichloroacetic acid, salicylic acid and trifluoroacetic acid.

When present, the amount of inhibitor may range from 10 ppm to 20% by weight, such as, for example, from 10 ppm to 5% or from 10 ppm to 1% by weight, relative to the total weight of the composition.

The compositions in accordance with an embodiment of the present disclosure may also contain at least one agent usually used in cosmetics, for instance reducing agents, fatty substances, plasticizers, softeners, antifoams, moisturizers, pigments, clays, mineral fillers, UV-screening agents, mineral colloids, peptizers, solubilizing agents, fragrances, preserving agents, anionic, cationic, nonionic or amphoteric surfactants, fixing or non-fixing polymers, polyols, proteins, vitamins, direct dyes or oxidation dyes, nacreous agents, propellent gases, mineral or organic thickeners such as benzylidene sorbitol, and N-acylamino acids. These agents may optionally be encapsulated. The capsule may be of polycyanoacrylate type.

The hair treatment process in accordance with the disclosure comprises applying the composition described above to keratin materials, which may, in embodiments of the present disclosure, occur in the presence of a nucleophilic agent with or without heating.

In an embodiment of the present disclosure, the nucleophilic agents may be chosen from a molecular compound, an oligomer, a dendrimer and a polymer containing nucleophilic functions. In a non-limiting manner, nucleophilic functions that may be mentioned include the following functions: R₂N⁻, NH₂⁻, Ph₃C⁻, R₃C⁻, PhNH⁻, pyridine, ArS⁻, R—C≡C⁻, R⁻, SH, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, wherein Ph is a phenyl group; Ar is an aryl group and R is chosen from a C₁-C₁₀ alkyl group.

In one embodiment of the present disclosure, the nucleophilic agent is water. This water may be provided by wetting beforehand.

It is also possible, in order to modify the reaction kinetics, to wet the keratin materials beforehand using an aqueous solution whose pH has been adjusted using a base, an acid or an acid/base mixture. The acid and/or the base may be mineral or organic.

These two operations may also be performed after applying the composition.

It is also possible to modify the anionic polymerization kinetics by preimpregnating the keratin materials with a nucleophilic agent. The nucleophilic agent may be used pure, as a solution, in the form of an emulsion, or may be encapsulated.

Nucleophilic agents capable of initiating the anionic polymerization are systems that are known per se, which are capable of generating a carbanion on contact with a nucleophilic agent, such as the hydroxyl ions contained in water. The term “carbanion” means the chemical species defined in “Advanced Organic Chemistry”, Third Edition, by Jerry March, page 141.

To modify the anionic polymerization kinetics, it is also possible to increase the nucleophilicity of the fiber via chemical conversion of the keratin material.

Non-limiting examples that may be mentioned include the reduction of the disulfide bridges of which keratin is partly composed, into thiols, before applying the composition of the disclosure. In a non-exhaustive manner, as reducing agents for the disulfide bridges of which keratin is partially composed, mention may be made of the following compounds:
anhydrous sodium thiosulfate;
powdered sodium metabisulfite;
thiourea;
ammonium sulfite;
thioglycolic acid;
thiolactic acid;
ammonium thiolactate;
glyceryl monothioglycolate;
ammonium thioglycolate;
thioglycerol;
2,5-dihydroxybenzoic acid;
diammonium dithioglycolate;
strontium thioglycolate;
calcium thioglycolate;
zinc formosulfoxylate;
isooctyl thioglycolate;
dl-cysteine; and
monoethanolamine thioglycolate.

In one embodiment of the present disclosure, to modify the anionic polymerization kinetics, and to reduce the rate of polymerization of the monomers of the invention, it may be possible to increase the viscosity of the composition. To do this, one or more polymers that have no reactivity towards the monomers in accordance with the invention may be added to the composition of the invention. In this context, mention may be made, in a non-exhaustive manner, of poly(methyl methacrylate) (PMMA) or alternatively cyanoacrylate-based copolymers as described in U.S. Pat. No. 6,224,622.

In one embodiment, the adhesion of the poly(cyanoacrylate) formed in situ may be improved by pretreating the fiber with polymers of any type, or a hair treatment may be performed before applying the composition of the invention, for instance a direct dyeing or oxidation dyeing, permanent-waving or hair relaxing operation.

The application of the compositions may or may not be followed by rinsing. The compositions of the present disclosure may be in various forms, such as, for example, in the form of lotions, sprays or mousses, and may be applied in the form of a shampoo or a hair conditioner.

The process may include a step of applying to the keratin materials at least one non-silicone polymer as defined above and a step of applying to the keratin materials at least one electrophilic monomer, the order of the steps being irrelevant.

In one embodiment, the application of the at least one non-silicone polymer is performed before applying the at least one electrophilic monomer.

According to the present disclosure, the monomers may be chosen from monomers capable of polymerizing on keratin fibers under cosmetically acceptable conditions. In one embodiment, the polymerization of the monomer can be performed at a temperature of less than or equal to 80° C., such as, for example, from 10 and 80° C. or from from 20 to 80° C., which does not prevent the application from being terminated by drying under a hood, blow-drying or treating with a flat iron or a crimping iron.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention are approximations, the numerical values set forth in the specific examples are reported as precisely as possible. Any numerical value, however, inherently contains certain errors necessarily resulting from the standard deviation found in its respective testing measurement.

The examples that follow are intended to illustrate the invention without, however, being limiting in nature.

In the examples, the percentages are expressed as weight percentages of active material.

### EXAMPLE 1

The following composition was prepared:

| | |
|---|---|
| n-Octyl 2-cyanoacrylate (1) | 60% |
| Acrylic/ethyl acrylate/acrylonitrile polymer sold under the name | 39% |
| Hystrech V-29 by the company Noveon | |
| Monoethanolamine | 1% |

(1) Rite Lok CON895, sold by the company Chemence

The maximum peel force, measured as indicated above, was 4 N, and the separation energy was 200 μJ.

The composition of the present disclosure made it possible, after several shampoo washes, to maintain the softness, sheen and hold of the hair, without it being necessary to reapply the composition.

### EXAMPLE 2

The following composition was prepared:

| | |
|---|---|
| Methylheptyl cyanoacrylate (1) | 60% |
| Acrylic/ethyl acrylate/acrylonitrile polymer | 39% |
| sold under the name Hystrech V-29 by the | |
| company Noveon | |
| Monoethanolamine | 1% |

(1) sold by the company Chemence

### EXAMPLE 3

The following composition was prepared:

| | |
|---|---|
| Ethoxyethyl cyanoacrylate (1) | 60% |
| Acrylic/ethyl acrylate/acrylonitrile polymer sold under the name | 39% |
| Hystrech V-29 by the company Noveon | |
| Monoethanolamine | 1% |

(1) EO 460 sold by the company Tong Shen

### EXAMPLE 4

The following composition was prepared:

| | |
|---|---|
| Butyl cyanoacrylate (1) | 60% |
| Acrylic/ethyl acrylate/acrylonitrile polymer | 39% |
| sold under the name Hystrech V-29 by the | |
| company Noveon | |
| Monoethanolamine | 1% |

(1) B 60 sold by the company Tong Shen

### EXAMPLE 5

The following composition was prepared:

| | |
|---|---|
| Ethylhexyl cyanoacrylate (1) | 60% |
| Acrylic/ethyl acrylate/acrylonitrile polymer sold under the name | 39% |
| Hystrech V-29 by the company Noveon | |
| Monoethanolamine | 1% |

(1) O-60 sold by the company Tong Shen

### EXAMPLE 6

The following composition was prepared:

| | |
|---|---|
| Methylheptyl cyanoacrylate (1) | 54% |
| Ethylhexyl cyanoacrylate (2) | 6% |
| Acrylic/ethyl acrylate/acrylonitrile polymer sold under the name | 39% |
| Hystrech V-29 by the company Noveon | |
| Monoethanolamine | 1% |

(1) sold by the company Chemence
(2) O-60 sold by the company Tong Shen

### EXAMPLE 7

The following composition was prepared:

| | |
|---|---|
| Methylheptyl cyanoacrylate (1) | 42% |
| Butyl cyanoacrylate (2) | 18% |
| Acrylic/ethyl acrylate/acrylonitrile polymer sold under the name | 39% |
| Hystrech V-29 by the company Noveon | |
| Monoethanolamine | 1% |

(1) sold by the company Chemence
(2) B-60 sold by the company Tong Shen

What is claimed is:

1. A method for the cosmetic treatment of keratin materials comprising applying a composition to the keratin materials, wherein the composition comprises, in a cosmetically acceptable medium:
at least one electrophilic monomer; and
at least one non-silicone polymer;
wherein the non-silicone polymer is chosen such that the composition gives, after drying, a film with a maximum peel force of greater than 1 newton.

2. The method according to claim 1, wherein the at least one non-silicone polymer is chosen from non-silicone fixing polymers.

3. The method according to claim 2, wherein the at least one non-silicone fixing polymer is chosen from cationic, anionic, amphoteric and nonionic non-silicone fixing polymers.

4. The method according to claim 3, wherein the cationic non-silicone fixing polymers are chosen from homopolymers or copolymers of acrylic esters, methacrylic esters, or amides containing amino functions; cationic polysaccharides; quaternary copolymers of vinylpyrrolidone; quaternary copolymers of vinylimidazole; and chitosans.

5. The method according to claim 3, wherein the anionic non-silicone fixing polymers are chosen from homopolymers or copolymers of acrylic acid or methacrylic acid, or salts thereof; crotonic acid copolymers; copolymers of C₄-C₈ monounsaturated carboxylic acids or anhydrides; polyacrylamides containing carboxylate groups; homopolymers or copolymers containing sulfonic groups; and anionic polyurethanes.

6. The method according to claim 3, wherein the amphoteric non-silicone fixing polymers are chosen from copolymers containing acidic vinyl units and containing basic vinyl units; crosslinked and acylated polyamino amides; polymers containing zwitterionic units; chitosan-based polymers; modified (C₁-C₅)alkyl vinyl ether/maleic anhydride copolymers; and amphoteric polyurethanes.

7. The method according to claim 3, wherein the nonionic non-silicone fixing polymers are chosen from polyalkyloxazolines; vinyl acetate homopolymers and copolymers; acrylic ester homopolymers and copolymers; acrylonitrile copolymers; styrene homopolymers and copolymers; polyamides; vinyllactam homopolymers other than vinylpyrrolidone homopolymers; vinyllactam copolymes; and nonionic polyurethanes.

8. The method according to claim 1, wherein the at least one electrophilic monomer is chosen from the compounds of formula (I): 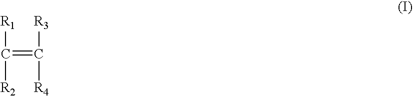
wherein:
R₁ and R₂ comprise, independently of each other, a sparingly or non-electron-withdrawing group chosen from:
a hydrogen atom,
a saturated or unsaturated, linear, branched or cyclic hydrocarbon-based group containing from 1 to 20 carbon atoms, and optionally containing at least one nitrogen, oxygen or sulfur atoms, and optionally substituted with at least one groups chosen from —OR, —COOR, —COR, —SH, —SR and —OH, and halogen atoms,
a modified or unmodified polyorganosiloxane residue,
a polyoxyalkylene group,
R₃ and R₄ comprise, independently of each other, an electron-withdrawing group chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups; linear or branched alkenyl groups; linear or branched alkynyl groups; C₁-C₄ mono- or polyfluoroalkyl groups; aryl groups; and aryloxy groups,
wherein R is chosen from a saturated or unsaturated, linear, branched or cyclic hydrocarbon-based group containing from 1 to 20 carbon atoms, optionally containing at least one nitrogen, oxygen or sulfur atom, and optionally substituted with at least one group chosen from —OR′, —COOR′, —COR′, —SH, —SR′ and —OH; halogen atoms; and a polymer residue obtained by free-radical polymerization, by polycondensation or by ring-opening;
wherein R′ is chosen from C₁-C₁₀ alkyl radicals.

9. The method according to claim 8, wherein the at least one electrophilic monomer is chosen from the compounds of formula (II): 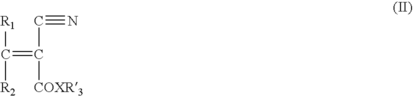
wherein:
X is chosen from NH, S, and O;
R₁ and R₂ comprise, independently of each other, a sparingly or non-electron-withdrawing group chosen from:
a hydrogen atom;
a saturated or unsaturated, linear, branched or cyclic hydrocarbon-based group containing from 1 to 20 carbon atoms, optionally containing at least one nitrogen, oxygen or sulfur atom, and optionally substituted with at least one group chosen from —OR, —COOR, —COR, —SH, —SR and —OH, and halogen atoms;
a modified or unmodified polyorganosiloxane residue; and
a polyoxyalkylene group;
R′₃ is chosen from a hydrogen atom and a radical R,
wherein R is chosen from a saturated or unsaturated, linear, branched or cyclic hydrocarbon-based group containing from 1 to 20 carbon atoms, optionally containing at least one nitrogen, oxygen or sulfur atom, and optionally substituted with at least one groups chosen from —OR′, —COOR′, —COR′, —SH, —SR′ and —OH; halogen atoms; and a polymer residue obtained by free-radical polymerization, by polycondensation or by ring-opening.

10. The method according to claim 9, wherein the at least one electrophilic monomer is chosen from C₁-C₂₀ polyfluoroalkyl 2-cyanoacrylates, (C₁-C₁₀)alkyl cyanoacrylates, and (C₁-C₄ alkoxy)(C₁-C₁₀ alkyl)cyanoacrylates.

11. The method according to claim 10, wherein the at least one electrophilic monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

12. The method according to claim 9, wherein the at least one electrophilic monomer is chosen from the compounds of formula (III): 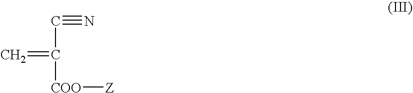
wherein:
Z is chosen from:
—(CH₂)₇—CH₃;
—CH(CH₃)—(CH₂)₅—CH₃;
—CH₂—CH(C₂H₅)—(CH₂)₃—CH₃;
—(CH₂)₅—CH(CH₃)—CH₃; and
—(CH₂)₄—CH(C₂H₅)—CH₃.

13. The method according to claim 1, wherein the at least one electrophilic monomer is present in the composition in an amount ranging from 0.001% to 80% by weight relative to the total weight of the composition.

14. The method according to claim 13, wherein the at least one electrophilic monomer is present in the composition in an amount ranging from 0.1% and 40% by weight relative to the total weight of the composition.

15. The method according to claim 14, wherein the at least one electrophilic monomer is present in the composition in an amount ranging from 1% and 20% by weight relative to the total weight of the composition.

16. The method according to claim 1, wherein the at least one electrophilic monomer is covalently bonded to supports.

17. The method according to claim 16, wherein the supports are chosen from polymers, oligomers or dendrimers.

18. The method according to claim 1, wherein the cosmetically acceptable medium is anhydrous.

19. The method according to claim 18, wherein the cosmetically acceptable medium is chosen from organic oils, silicones, mineral oils, plant oils, waxes, C₅-C₁₀ alkanes, acetone, methyl ethyl ketone, esters of C₁-C₂₀ acids, esters of C₁-C₈ alcohols, dimethoxyethane, diethoxyethane, C₁₀-C₃₀ fatty alcohols, C₁₀-C₃₀ fatty acids, C₁₀-C₃₀ fatty amides and C₁₀-C₃₀ fatty alkyl esters, and mixtures thereof.

20. The method according to claim 1, wherein the composition further comprises at least one polymerization inhibitor.

21. The method according to claim 20, wherein the at least one polymerization inhibitor is an anionic and/or free-radical polymerization inhibitor.

22. The method according to claim 20, wherein the at least one polymerization inhibitor is chosen from sulfur dioxide, nitric oxide, lactone, boron trifluoride, hydroquinone and derivatives thereof, tert-butylhydroquinone (TBHQ), benzoquinone and derivatives thereof, catechol and derivatives thereof, anisole and derivatives thereof, pyrogallol, 2,4-dinitrophenol, 2,4,6-trihydroxybenzene, p-methoxyphenol, hydroxybutyltoluene, alkyl sulfates, alkyl sulfites, alkyl sulfones, alkyl sulfoxides, alkyl sulfides, mercaptans and 3-sulfonene.

23. The method according to claim 22, wherein the at least one polymerization inhibitor is chosen from hydroquinone monoethyl ether.

24. The method according to claim 22, wherein the at least one polymerization inhibitor is chosen from duroquinone.

25. The method according to claim 22, wherein the at least one polymerization inhibitor is chosen from t-butylcatechol and methoxycatechol.

26. The method according to claim 22, wherein the at least one polymerization inhibitor is chosen from methoxyanisole, hydroxyanisole and butylhydroxyanisole.

27. The method according to claim 20, wherein the at least one polymerization inhibitor is present in an amount ranging from 10 ppm to 20% by weight relative to the total weight of the composition.

28. The method according to claim 27, wherein the at least one polymerization inhibitor is present in an amount ranging from 10 ppm to 5% by weight relative to the total weight of the composition.

29. The method according to claim 28, wherein the at least one polymerization inhibitor is present in an amount ranging from 10 ppm to 1% by weight relative to the total weight of the composition.

30. The method according to claim 1, wherein the at least one non-silicone polymer is present in the composition in an amount ranging from 0.05% and 99% by weight relative to the total weight of the composition.

31. The method according to claim 30, wherein the at least one non-silicone polymer is present in the composition in an amount ranging from 0.1% to 95% by weight relative to the total weight of the composition.

32. The method according to claim 1, wherein the at least one non-silicone polymer is present in the composition in an amount ranging from 0.2% to 30% by weight relative to the total weight of the composition.

33. The method according to claim 1, wherein the composition further comprises at least one agent chosen from reducing agents; fatty substances; plasticizers; softeners; antifoams; moisturizers; pigments; clays; mineral fillers; UV-screening agents; mineral colloids; peptizers; solubilizing agents; fragrances; preserving agents; anionic, cationic, nonionic or amphoteric surfactants; fixing or non-fixing polymers; polyols; proteins; vitamins; direct dyes or oxidation dyes; nacreous agents; propellent gases; and mineral or organic thickeners.

34. The method according to claim 33, wherein the at least one agent is encapsulated.

35. The method according to claim 1, wherein the keratin materials are chosen from hair, eyelashes and nails.

36. The method according to claim 1, wherein the composition is in a form chosen from a lotion, a spray and a mousse.

37. A cosmetic composition, comprising, in a cosmetically acceptable medium, at least one non-silicone fixing polymer and at least one electrophilic monomer chosen from the compounds of formula (I): 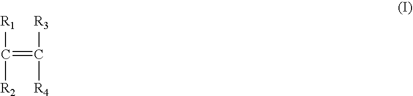
wherein:
R₁ and R₂ comprise, independently of each other, a sparingly or non-electron-withdrawing group chosen from:
a hydrogen atom,
a saturated or unsaturated, linear, branched or cyclic hydrocarbon-based group containing from 1 to 20 carbon atoms, and optionally containing at least one nitrogen, oxygen or sulfur atoms, and optionally substituted with at least one group chosen from —OR, —COOR, —COR, —SH, —SR and —OH, and halogen atoms,
a modified or unmodified polyorganosiloxane residue,
a polyoxyalkylene group,
R₃ and R₄ comprise, independently of each other, an electron—withdrawing group chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups; linear or branched alkenyl groups; linear or branched alkynyl groups; C₁-C₄ mono- or polyfluoroalkyl groups; aryl groups; and aryloxy groups;
wherein R is chosen from a saturated or unsaturated, linear, branched or cyclic hydrocarbon-based group containing from 1 to 20 carbon atoms, optionally containing at least one nitrogen, oxygen or sulfur atom, and optionally substituted with at least one group chosen from —OR′, —COOR′, —COR′, —SH, —SR′ and —OH; halogen atoms; and a polymer residue obtained by free-radical polymerization, by polycondensation or by ring-opening;
wherein R′ is chosen from C₁-C₁₀ alkyl radicals.

38. The composition according to claim 37, wherein the at least one non-silicone fixing polymer is chosen from cationic, anionic, amphoteric and nonionic non-silicone fixing polymers, and mixtures thereof.

39. A process for treating keratin materials, comprising:
applying to the keratin materials at least one composition comprising at least one non-silicone polymer, wherein the non-silicone polymer is chosen such that the composition gives, after drying, a film with a maximum peel force of greater than 1 newton; and
applying to the keratin materials at least one electrophilic monomer.

40. The process according to claim 39, wherein the application of the at least one non-silicone polymer is performed before the application of the at least one electrophilic monomer.

41. The process according to claim 39, wherein the composition is applied to the keratin materials in the presence of a nucleophilic agent.

42. The process according to claim 41, wherein the nucleophilic agent is chosen from molecular compounds, oligomers, dendrimers or polymers containing nucleophilic functions chosen from: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O;
wherein Ph is a phenyl group, Ar is an aryl group, and R is a C₁-C₁₀ aryl group.

43. The process according to claim 41, wherein the nucleophilic agent is water.

44. The process according to claim 41, wherein the composition is applied to the keratin materials, which have been wetted beforehand using an aqueous solution having a pH adjusted using a base, an acid or an acid/base mixture.

45. The process according to claim 41, wherein the keratin materials are preimpregnated with a nucleophilic agent other than water.

46. The process according to claim 41, wherein the keratin materials are reduced before applying the composition.

47. The process according to claim 41, wherein the application of the composition is followed by rinsing.

48. The process according to claim 41, wherein the keratin materials are hair.

49. A kit comprising:
a first composition containing at least one electrophilic monomer and optionally at least one anionic and/or free-radical polymerization inhibitor; and
a second composition comprising, in a cosmetically acceptable medium, at least one non-silicone polymer, wherein the non-silicone polymer is chosen such that the composition gives, after drying, a film with a maximum peel force of greater than 1 newton.
